SEQUENCE LISTING (25 sequences)

SEQ ID NO: 1 - DNA, 50 bp, Mus sp.
  source 1..50: mol_type = genomic DNA; note = Mouse
acatttgtgtcttcattaccaggaaatattaagggacattgtgccatcta

SEQ ID NO: 2 - DNA, 50 bp, Rattus sp.; identical to SEQ ID NO: 1
  source 1..50: mol_type = genomic DNA; note = Rat

SEQ ID NO: 3 - DNA, 50 bp, Oryctolagus sp.; identical to SEQ ID NO: 1
  source 1..50: mol_type = genomic DNA; note = Rabbit

SEQ ID NO: 4 - DNA, 50 bp, Homo sapiens; identical to SEQ ID NO: 1
  source 1..50: mol_type = genomic DNA; note = Human

SEQ ID NO: 5 - DNA, 50 bp, Tupaia sp.; identical to SEQ ID NO: 1
  source 1..50: mol_type = genomic DNA; note = Tree shrew

SEQ ID NO: 6 - DNA, 50 bp, Canis sp.; identical to SEQ ID NO: 1
  source 1..50: mol_type = genomic DNA; note = Dog

SEQ ID NO: 7 - DNA, 50 bp, Sorex sp.; identical to SEQ ID NO: 1
  source 1..50: mol_type = genomic DNA; note = Shrew

SEQ ID NO: 8 - DNA, 50 bp, Loxodonta sp.; identical to SEQ ID NO: 1
  source 1..50: mol_type = genomic DNA; note = Elephant

SEQ ID NO: 9 - DNA, 50 bp, Didelphis sp.; identical to SEQ ID NO: 1
  source 1..50: mol_type = genomic DNA; note = Opossum

SEQ ID NO: 10 - DNA, 50 bp, Ornithorhynchus sp.; identical to SEQ ID NO: 1
  source 1..50: mol_type = genomic DNA; note = Platypus

SEQ ID NO: 11 - DNA, 50 bp, Gallus gallus; identical to SEQ ID NO: 1
  source 1..50: mol_type = genomic DNA; note = Chicken

SEQ ID NO: 12 - DNA, 26 bp, Bacillus thuringiensis
  source 1..26: mol_type = genomic DNA
gtaaacggcggccgtaactataacgg

SEQ ID NO: 13 - DNA, 26 bp, Bacillus anthracis
  source 1..26: mol_type = genomic DNA
gtaaacggcggccgtaactataacgg

SEQ ID NO: 14 - DNA, 26 bp, Streptococcus plurextorum
  source 1..26: mol_type = genomic DNA
gtaaacggcggccgtaactataacgg

SEQ ID NO: 15 - DNA, 26 bp, Escherechia coli
  source 1..26: mol_type = genomic DNA
gtaaacggcggccgtaactataacgg

SEQ ID NO: 16 - DNA, 26 bp, Esherechia coli 2
  source 1..26: mol_type = genomic DNA
gtaaacggcggccgtaactataacgg

SEQ ID NO: 17 - DNA, 26 bp, Salmonella enterica
  source 1..26: mol_type = genomic DNA
gtaaacggcggccgtaactataacgg

SEQ ID NO: 18 - DNA, 26 bp, Staphylococcus aureus
  source 1..26: mol_type = genomic DNA
gtaaacggcggccgtaactataacgg

SEQ ID NO: 19 - DNA, 26 bp, Pseudomonas aeruginosa
  source 1..26: mol_type = genomic DNA
gtaaacggcggccgtaactataacgg

SEQ ID NO: 20 - DNA, 26 bp, Mycoplasma hominis
  source 1..26: mol_type = genomic DNA
gtgaacggcggccgtaactataacgg

SEQ ID NO: 21 - DNA, 26 bp, Mycoplasma bovis
  source 1..26: mol_type = genomic DNA
gtgaacggcggccgtaactataacgg

SEQ ID NO: 22 - DNA, 26 bp, Mycoplasma arginini
  source 1..26: mol_type = genomic DNA
gtgaacggcggccgtaactataacgg

SEQ ID NO: 23 - DNA, 26 bp, Mycoplasma hyorhinis
  source 1..26: mol_type = genomic DNA
gtgaacggcggccgtaactataacgg

SEQ ID NO: 24 - DNA, 26 bp, Mycoplasma fermentans
  source 1..26: mol_type = genomic DNA
gtgaacggcggccgtaactataacgg

SEQ ID NO: 25 - DNA, 26 bp, Mycoplasma orale
  source 1..26: mol_type = genomic DNA
gtgaacggcggccgtaactataacgg

(19) United States
(12) Patent Application Publication
(10) Pub. No.: US 2026/0110031 A1
(43) Pub. Date: Apr. 23, 2026

(54) METHODS FOR EVALUATING HEALTH AND STABILITY OF CULTURED CELLS

(71) Applicant: THE BOARD OF TRUSTEES OF THE LELAND STANFORD JUNIOR UNIVERSITY, Stanford, CA (US)

(72) Inventors: Fabian Patrik Suchy, Stanford, CA (US); Hiromitsu Nakauchi, Stanford, CA (US); Ian Hsu, Stanford, CA (US)

(21) Appl. No.: 19/116,672

(22) PCT Filed: Oct. 13, 2023
(86) PCT No.: PCT/US2023/076824

§ 371 Date: Mar. 28, 2025

Related U.S. Application Data
(60) Provisional application No. 63/416,151, filed on Oct. 14, 2022.

(51) Int. Cl. C12Q 1/6883; C12Q 1/689

(52) U.S. Cl. CPC C12Q 1/6883; C12Q 1/689; C12Q 2600/156

(57) ABSTRACT

The present invention provides a set of robust, genetic-based assays to screen for common abnormalities that occur in cultured cells utilizing a digital PCR (dPCR) platform.

### BACKGROUND

Stem cells have diverse utility in basic, agricultural, and clinical research including applications in the modeling of complex diseases, creating transgenic animals, gene therapy, cell therapy, regenerating lost tissue, and organ biogenesis. Stem cells of particular clinical interest include both human induced pluripotent stem cells (iPSCs), and hematopoietic stem cells (HSCs). However, regardless of cell-type, various genetic aberrations are known to accumulate over time in cultured cells, some resulting in phenotypic changes and irregularities resulting in, among other disadvantages, irreproducible preclinical data which raises the costs of developing new therapeutics based on cultured cells, including stem cells. Most large karyotypic abnormalities occur as gain or loss of an entire chromosome/arm. These have been extensively characterized in primary cells but are likely to occur in all dividing cells in vitro. Recently, specific oncogenic mutations that frequently occur in cancer cells in vivo have also been documented in human stem cell culture. Particularly, TP53 mutations, which occur in 20% of cancers, were found to enhance reprogramming and gene editing in human iPSCs. These mutations often occur at the same hot-spot sites. Often these large and small genomic abnormalities are undertested due to the complexity of such tests using current technologies. For example, microscopic karyotyping is usually done only once (by an expert), immediately following cell-line establishment, yet chromosomal abnormalities are almost guaranteed to appear after extended culture. In contrast, detection of oncogenic mutations is most frequently performed on an entirely different platform, usually Sanger or next-generation sequencing.

In addition, contamination of cultured cells with bacteria, including Mycoplasma occur at an alarmingly high rates. Such infections can increase cell stress, induce DNA damage, and change protein expression. Since Mycoplasma is resistant to common antibiotics such as penicillin/streptomycin and cannot be detected by standard light microscopy, Mycoplasma infections often spread undetected. Contamination with other cells cultured in the same facility may also occur and be difficult to detect. For example, HeLa cells, one of the most common cell-lines in the world, can contaminate adjacent cell cultures by aerosol transmission. As a cancer line, HeLa cells can outgrow other cells types and may not be easily detected if the cell morphology is similar. It is estimated that over 30,000 publications have used misidentified lines. Despite the magnitude of the problem, most researchers do not frequently authenticate their cell lines because the process is complex, time consuming, and expensive as it may involve multiple different assays including morphological analysis, isoenzymology, and short-tandem repeat (STR) analysis via capillary electrophoresis.

Better methods for evaluating the integrity and genetic stability of cultured cells are urgently needed. The present invention addresses this need.

### BRIEF SUMMARY

The present invention provides multiplex digital PCR-based methods for screening for common abnormalities in cultured cells, including aneuploidy, genetic mutations, and Mycoplasma contamination, as well as methods for cell line verification and related compositions and methods.

In one aspect, the invention provides a method for detecting aneuploidy in a plurality of cells, the method comprising determining a ratio of an amount of each chromosome in a genome of the cells, the method comprising contacting DNA from the cells with an enzyme mixture and one or more chromosomal primer pairs to produce a reaction mixture, where each chromosomal primer pair is directed to a different template DNA sequence on a target chromosome of the genome, preferably where the template sequences are located at opposite distal ends of the target chromosome from each other, subjecting the reaction mixture to a digital PCR process including partitioning the reaction mixture and subjecting the partitioned mixture to PCR amplification to produce amplification products, detecting the amplification products in the reaction mixture, determining an amount of amplification product for each target chromosome, and determining a ratio of the amount of amplification product for each target chromosome in the sample, where a ratio other than 2:2 for a target chromosome indicates aneuploidy.

The method may also include where detecting the amplification products in the reaction mixture includes detecting a fluorescent signal, optionally at least two different fluorescent signals.

In embodiments, the amplification products of the chromosomal primer pairs are labelled with at least two different detectable labels.

In embodiments, the one or more chromosomal primer pairs includes from 1 to 12, from 3 to 6, from 6-9, or from 9-12 primer pairs, optionally where each primer pair in the reaction mixture is directed to a different target chromosome.

In embodiments, the one or more chromosomal primer pairs contains a detectable label, optionally where two or more chromosomal primer pairs contain at least two different detectable labels.

In embodiments, the one or more chromosomal primer pairs is unlabeled and the reaction mixture includes one or more DNA probes, each includes a detectable label, optionally where two or more DNA probes contain at least two different detectable labels.

In embodiments, the enzyme mixture comprises a DNA polymerase, a source of divalent cations, a mixture of deoxynucleotide triphosphates (dNTPs), and a suitable buffering system.

In embodiments, the method is performed by contacting a plurality of samples comprising DNA from the cells with a plurality of enzyme mixtures including one or more chromosomal primer pairs to produce a plurality of reaction mixtures.

In embodiments, the template sequences of each target chromosome share at least 70%, preferably at least 90% sequence identity across human and non-human primate genomes; or at least 70%, preferably at least 90% sequence identity across human and mouse and/or rat genomes.

In embodiments, the detectable label includes a fluorophore or a fluorescent reporter-quencher pair.

In embodiments, the contacting takes place in a plurality of separate containers, each container includes a mixture and a plurality of chromosomal primer pairs.

In embodiments, each chromosomal primer pair in the same container is directed to a template sequence of a different chromosome.

In embodiments, the plurality of separate containers consists of from 5-15 containers.

In embodiments, the plurality of separate containers comprise a microtiter plate.

In embodiments, two or more of the template sequences comprise at least one genetic mutation selected from a single nucleotide polymorphism (SNP) or a point mutation in a gene. In embodiments, the at least one genetic mutation includes an SNP and the method includes measuring a concentration of the SNP based on the amplification products in the reaction mixture and determining a corresponding haplotype for the cells. In embodiments, the at least one genetic mutation is a point mutation in a p53 gene, optionally where the genetic mutation is selected from the group consisting of G245C, G245S, R248W, R248Q, and R175H in a human p53 gene or corresponding mutation in a murine p53 gene.

In embodiments, the method may also include a further step of contacting a second sample comprising DNA from the cells with a further mixture comprising a set of unlabeled primers directed to a sequence having high homology across two or more bacterial genomes to form a Mycoplasma detection reaction mixture. In embodiments, the Mycoplasma detection reaction mixture includes a first and second DNA probe, the first DNA probe includes a first detectable label and the second DNA probe includes a second detectable label. In embodiments, the first and second DNA probes comprise the same sequence except for a single nucleotide mismatch, where the single nucleotide mismatch distinguishes Mycoplasma from other bacteria. In embodiments, the method further includes detecting Mycoplasma contamination in the cells based on detecting signals from the first and second DNA probes.

Other technical features may be readily apparent to one skilled in the art from the following figures, descriptions, and claims.

### BRIEF DESCRIPTION OF THE DRAWINGS

FIG. 1 is a schematic illustration of a streamlined assay for screening for common abnormalities in cultured cells in accordance with one embodiment.

FIG. 2A illustrates the location of 42 primer/probe sets targeting mouse chromosomes. Green and purple differentiate between the two sets designed for each chromosome.

FIG. 2B illustrates a 2D digital PCR blot of 6 primer/probe sets multiplexed in a single reaction mixture. Neg=negative cluster. 1-6 indicate clusters corresponding to mouse chromosome 1-6.

FIG. 2C illustrates in a tabular format the reactions used to count each mouse chromosome twice. Each reaction takes place in a separate container, for example a separate well of a microtiter plate. Number indicates the chromosome targeted by the primer/probe sets. Colors correspond to FIG. 2A.

FIG. 2D is a bar graph depicting the results of 7 reactions shown in FIG. 2C run on male mouse DNA.

FIG. 2E illustrates an example of an ultra-conserved region in mammalian genome.

FIG. 2F illustrates primer/probe sets designed for mouse and mapped to human chromosomes in accordance with one embodiment.

FIG. 2G is a bar graph depicting the results of 7 reactions shown in FIG. 2C run on male human DNA.

FIG. 3A illustrates a 2D digital PCR blot of three ddPCR-SND primer/probe sets multiplexed. The source data is “person 3” indicated in FIG. 3B. Neg=negative cluster. The first number in front of labelled clusters indicates the targeted chromosome. The second number indicates variant 1 or 2.

FIG. 3B is a bar graph depicting the results of ddPCR-SND assay after running on DNA extracted from three different individuals.

FIG. 4A illustrates an ultra-conserved region of bacterial ribosomal RNA. Highlighted A residues indicate where non-Mycoplasma genomes differ from most Mycoplasma genomes. Mycoplasma-specific and bacteria-specific probes were designed in this region.

FIG. 4B is a bar graph depicting the results of ddPCR-SND Mycoplasma/bacteria detection assay on DNA extracted from multiple cell lines including fibroblasts, embryonic stem cells (ESC), and induced pluripotent stem cells (iPSC).

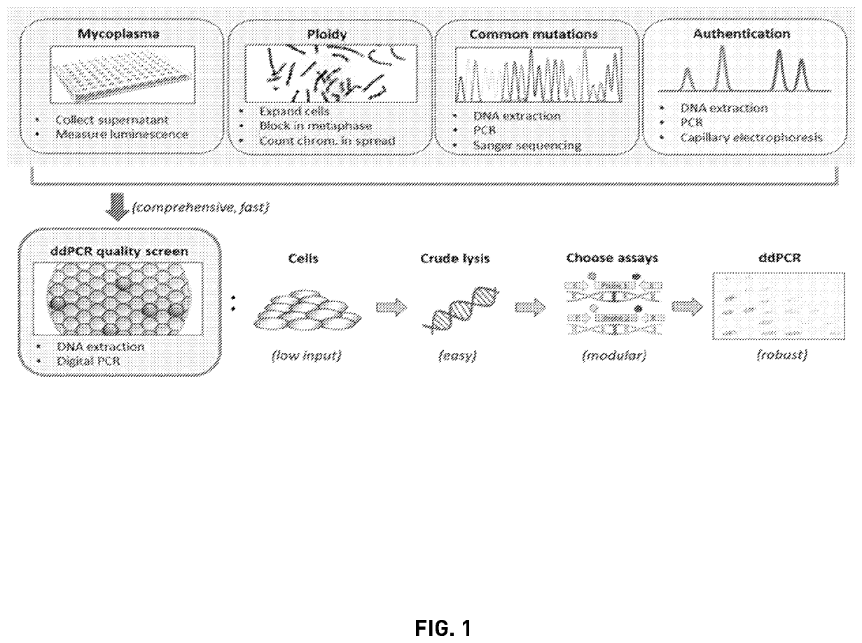

### DETAILED DESCRIPTION

The present invention provides a set of robust, genetic-based assays to screen for common abnormalities that occur in cultured cells. The assays described here are performed on a single platform utilizing digital PCR, for example droplet digital PCR (ddPCR). ddPCR is particularly advantageous due to its quantitative power, high-throughput capabilities, resistance to inhibitors, and reproducibility. Further advantageously, the assays described here can be performed with small sample sizes, for example less than 50,000 cells, and results can be acquired within hours, enabling affordable ongoing quality assurance of cultured cells.

As used herein, the term “primer” refers to an oligonucleotide comprising a sequence complementary to a sequence of a target nucleic acid, which may also be referred to as the “template” sequence, such that at least a portion of the primer anneals to the template sequence of the target nucleic acid and serves as a point of initiation for synthesis of a primer extension product under suitable conditions for such a reaction. The primer extension product may also be referred to as “the amplification product” or “the PCR product”. Suitable conditions include conditions for a digital PCR reaction or a droplet digital PCR reaction.

Generally PCR amplification primers are provided as primer pairs, wherein each primer pair consists of a first primer having a sequence that is at least partially complementary to a 5′ flanking region of the template sequence and a second primer having a sequence that is at least partially complementary to a 3′ flanking region of the template sequence. In embodiments, the primer may be a degenerate or promiscuous primer. In embodiments of the methods described here, the template sequence is selected from a sequence of a chromosome, a sequence comprising a single nucleotide polymorphism, a sequence comprising a common genetic mutation found in cultured cells, such as a p53 mutation, and a sequence of bacteria, including a bacteria of the genus Mycoplasma. A primer may have a nucleotide length of from 10-50 nucleotides, from 10-40 nucleotides, from 10-30 nucleotides, or from 10-20 nucleotides.

In embodiments of the methods described here, a primer may be from about 15-40 or from about 15-30 nucleotides in length.

The amplification products of digital PCR reactions, including ddPCR, may be detected using any suitable method. For example, the PCR primers may be modified to include a detectable moiety, such as methyl group, a biotin or digoxigenin moiety, or a fluorescent molecule, to assist in detection of the amplification product. Suitable fluorescent molecules, which may also be referred to as fluorophores, include fluorescein isothiocyanate (FITC), phycoerythrin (PE), cyanine (Cy3), VIC fluorescent dye, FAM (6-carboxyfluorescein), hexachlorofluorescein (HEX) and indocyanine (Cy5).

Unlabelled PCR primers may also be paired with a labelled DNA probe. The term “probe” refers to an oligonucleotide that hybridizes with a target nucleic acid sequence. In embodiments, the probe is complementary to an internal segment of the amplification product. Generally, the melting point of a suitable probe will be 5-10° C. higher than the melting point of the primers present in the same PCR reaction, however it may be lower if necessary to aid in single-nucleotide discrimination assays.

Suitable probes may include a fluorescent label or a fluorescent reporter-quencher pair. Quenchers are known in the art and may include, for example, tetramethylrhodamine, and BLACK HOLE QUENCHERSup®/Sup (BHQ). An exemplary fluorescent reporter-quencher pair is fluorescein (FAM), which emits green fluorescence, and BHQ. Fluorescent reporter-quencher pairs are utilized, for example, in “hydrolysis probes” in which the fluorescent portion of the pair is released during extension by a polymerase having 5′ to 3′ exonuclease activity. Molecular beacons also utilize a reporter-quencher pair in which the stem-loop structure of the probe keeps the fluorophore and quencher together until the probe anneals to its target sequence. Other examples are described in Gudnason et al. (2007). Comparison of multiple DNA dyes for real-time PCR: Effects of dye concentration and sequence composition on DNA amplification and melting temperature. Nucleic Acids Res 35, e127.

In embodiments, the probe may comprise a pair of molecules that collectively exhibit fluorescence resonance energy transfer (FRET) when in sufficiently close proximity to one another. The pair of dyes may include two emitters, or an emitter and a quencher. In embodiments, fluorescence emission from a pair of dyes changes when the dyes are separated from one another, such as by cleavage of the probe during primer extension (e.g, a 5′ nuclease assay, such as with a TaqManSup™/Sup probe), or when the probe hybridizes to an amplicon (e.g, a molecular beacon probe). In accordance with this embodiment, the emission spectrum of one molecule overlaps the excitation spectrum of the other, resulting in “quenching” of the first fluorophore by the second unless PCR product is generated by the reaction. Where PCR product is generated, the probe is degraded via the 5′-nuclease activity of the polymerase, e.g., a Taq polymerase, allowing the two fluorophores to separate, reducing quenching and increasing the intensity of the fluorphores. In other exemplary embodiments, a probe and one of the primers of a primer pair may be combined in the same molecule (e.g, AmplifluorSup™/Sup primers or ScorpionsSup®/Sup primers). As an example, the primer-probe molecule may include a primer sequence at its 3′ end and a molecular beacon-style probe at its 5′ end. With this arrangement, related primer-probe molecules labeled with different fluorescent molecules can be used in a multiplexed assay with the same reverse primer to quantify target sequences differing by a single nucleotide (single nucleotide polymorphisms (SNPs)).

As used herein, the term “sequence identity” refers to the percentage match between two sequences, on a nucleotide-by-nucleotide basis, over a given window of comparison when the two sequences are optimally aligned. Generally the window of comparison comprises at least 15 contiguous nucleotide positions. Optimal alignment of sequences for purposes of determining sequence identity may be conducted using methods known in the art including the local homology algorithm of Smith and Waterman (Adv. Appl. Math. (1981) 2:482), the homology alignment algorithm of Needleman and Wunsch (J. Mol. Biol. (1970) 48:443), the search for similarity method of Pearson and Lipman (Proc. Natl. Acad. Sci. (U.S.A.) (1988) 85:2444), and computerized implementations of these algorithms which are widely available, for example the Basic Local Alignment Search Tool (BLAST) set of algorithms available through the US National Library of Medicine, National Center for Biotechnology Information web server.

### Digital PCR-Based Methods

The methods of the invention utilize detection and quantification of specific nucleic acid target sequences using a platform based on a digital polymerase chain reaction (“digital PCR” or “dPCR”). In general, digital PCR utilizes a combination of limiting dilution, end-point PCR, and Poisson statistics to determine an absolute measure of nucleic acid concentration in a sample. See Vogelstein and Kinzler in Proc. Natl. Acad. Sci. U.S.A. 1999, 96, 9236-9241. In practice, different approaches may be utilized to increase the number of replicates, or partitions, a sample is divided into for increased dynamic range of target DNA quantitation. For example, microwells or microfluidic chambers may be used to divide a sample into hundreds of nanoliter partitions; or in a method based on emulsion PCR, templates may be clonally amplified on beads; or a water-in-oil droplet approach may be used, for example as described by Hindson et al., Anal. Chem. 2011, 83, 22, 8604-8610, referred to herein as “droplet digital PCR” or “ddPCR”. Exemplary formats for the partitions of a dPCR reaction include microwell plates, also referred to as microtiter plates. Other suitable arrays of chambers include those of a microfluidic chip.

In exemplary embodiments, the methods of the invention utilize detection and quantification of specific nucleic acid target sequences using a droplet digital PCR (ddPCR) platform. In embodiments, performing a ddPCR reaction comprises steps of forming a PCR reaction mixture comprising target nucleic acid molecules and suitable primers and/or probes, generating a multiplicity of droplets from the PCR reaction mixture, subjecting the droplets to thermal cycling to amplify target nucleic acid molecules, and detecting the amplified target nucleic acid molecules which may also be referred to interchangeably herein as “PCR products”, “amplification products”, “reaction products”, or “ddPCR products”.

The methods may also comprise further steps of quantifying amplified target nucleic acids as well as additional analyses such as amplitude-based multiplexing or ratio-based multiplexing, described in more detail below in the section entitled “Aneuploidy Screen”.

The PCR reaction mixture generally comprises, in addition to the target nucleic acid molecules, a mixture of deoxynucleotide triphosphates (dNTPs), a DNA polymerase, and a suitable buffer comprising a buffering agent and a divalent cation, such as magnesium (Mg2+). In embodiments, the mixture of dNTPs does not include dUTP.

DNA polymerases are enzymes that perform a template-directed synthesis of nucleic acids such as DNA and are well-known in the art. Examples of commercially available polymerase enzymes include Taq DNA polymerase, 9° WM DNA polymerase, Deep Vent™ DNA polymerase, Manta DNA polymerase, Bst DNA polymerase, phi29 DNA polymerase, Takyon™ DNA polymerase (Eurogentec; EP2240576), AccuStart II Taq DNA (Quanta Bio).

In accordance with the exemplary methods described here, ddPCR reaction mixtures are typically from 20-30 microliters in volume. In embodiments, the step of generating a multiplicity of droplets from the PCR reaction mixture produces an average number of about 15,000 droplets, which may also be referred to as “partitions”. An exemplary thermal cycling reaction is 95° C.×10 minutes; 50 cycles of 94° C.×30 sec and 60° C.×60 sec; 98° C.×10 minutes; hold at 4° C.

Commercially available protocols and systems for performing ddPCR are available, for example the “QX” product line available from Bio-Rad Laboratories, Inc. Suitable systems for preparation ddPCR reactions and analysis of ddPCR reaction products in accordance with the methods described here include, for example, the QX200 ddPCR system (BioRad, Hercules, CA). Suitable data analysis packages include QuantaSoft Analysis Pro version 1.0.596 (BioRad, Hercules, CA).

In embodiments, the methods may comprise a step of droplet generation. In embodiments, droplet generation comprises partitioning the PCR reaction mixtures into water-in-oil droplets comprising an aqueous core containing the reaction mixture encapsulated within an oil envelope. The droplets may range in size from about 5-250 picometers (pm) based on average diameter, and may have a volume of about 1 picoliter (pl) to 1 nanoliter (nl). Droplets may be generated using microfluidic technology, for example utilizing a commercially available droplet generator such as the Bio-Rad system referenced above. Droplets may be transferred to a suitable format, such as a microtiter plate having a suitable number of wells, for amplification of target nucleic acid sequences by thermal cycling. After amplification, droplet fluorescence is measured by a droplet reader. Only droplets containing amplified target nucleic acid will generate a positive fluorescence reading. Counts of positive and negative droplets are related to the target's concentration in the sample by the Poisson distribution utilizing known methods, for example as described in Hindson et al., Anal. Chem. 2011, 83, 22, 8604-8610.

### Reaction Conditions

A digital PCR reaction in accordance with the methods described here is performed under standard conditions which may generally be described as a reaction mixture comprising a mixture of deoxynucleotide triphosphates (dNTPs), a DNA polymerase, a source of divalent cations, such as magnesium chloride, and a suitable buffering system. The polymerase reaction generally takes place at a temperature in the range of 55-60° C. which forms part of a thermocycle profile. Exemplary thermocycle profile conditions may be: 95° C.×10 minutes; 50 cycles of 94° C.×30 sec and 60° C.×60 sec; 98° C.×10 minutes; hold at 4° C.

### Droplet Target Sequence Selection

In general, the target nucleic acid sequence to be amplified is generally around 75-200 bp. The target sequence preferably has low secondary structure, as determined for example using software analytics of nucleic acid folding and hybridization, such available on the UNAFold web server hosted by Nicholas R. Markham, Leslie S. Zuker, and Michael Zuker. In addition, the target sequence preferably lacks repeats of single bases longer than 4 in a row and has a GC content in the range of 50-60%.

### Primer Design

In general, primers for digital PCR or ddPCR should have a guanidine to cytosine (“GC”) content in the range of 50-60% and a melting temperature between 50 and 65 C in a solution comprising 50 mM salt and 300 nM oligonucleotide. Melting temperature of oligonucleotide primers can be determined, for example, using the nearest neighbor method as described in Kibbe WA. ‘OligoCalc: an online oligonucleotide properties calculator’. (2007) Nucleic Acids Res. 35(webserver issue): May 25. General principles of primer design described in H. Erlich, PCR Technology, Principles and Application for DNA Amplification (1989). Primers may also be designed using software such as Primer3™ (ThermoFisher Scientific), described in Untergasser et al., Nucleic Acids Res. 2012 Aug. 1; 40(15):e115, and related software.

### Methods of Extracting/Isolating DNA for Use in Digital PCR Assay

An advantage of the ddPCR-based methods described here is that the nucleic acid sample does not need to be highly purified because ddPCR is less sensitive to the presence of cellular nucleases and other contaminants that may inhibit the reaction, compared for example to other PCR-based methods such as qPCR. Accordingly, in embodiments cells are lysed and the crude lysate is directly analyzed, further saving time and cost. This enables affordable, ongoing quality assurance of cultured cells. Any method for crude lysis may be used, including commercially available crude-lysis buffers. An exemplary crude lysis buffer may include 150 mM NaCl, 0.1% SDS, 15 mM Tris pH 8.0, 5 mM EDTA, and 0.2 mg/ml proteinase K. Cells lysed with this buffer would be heat treated for approximately 10 minutes at 55° C. followed by approximately 5 minutes at 80° C.

### Aneuploidy Screen

The present invention provides digital PCR or ddPCR-based methods for detecting aneuploidy in a population of cells. According to the methods described here, chromosome counting is achieved by designing PCR primer/probe sets targeting at least two different regions of each chromosome in the genome of the organism from which the cells are derived, referred to herein as “chromosomal primer pairs”. Preferably the at least two different regions are located at distal ends of the target chromosome, although this is not required. In a diploid genome, each chromosome will be amplified twice where the chromosomal primers consist of two primer pairs.

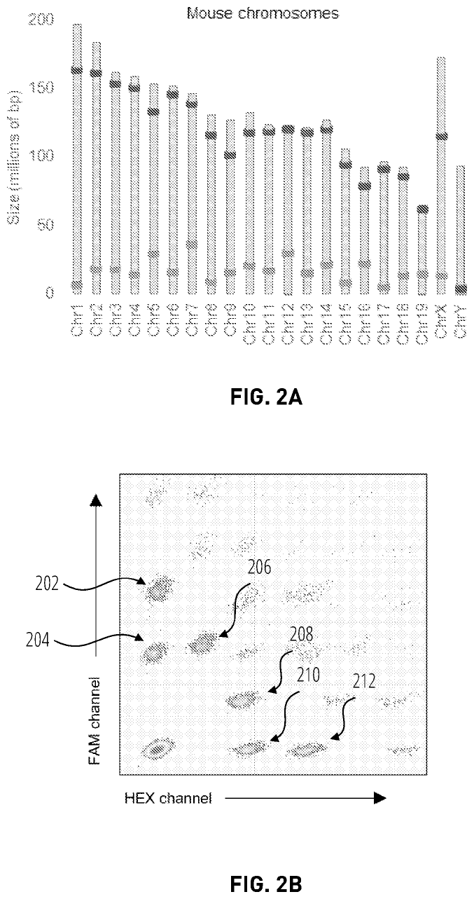

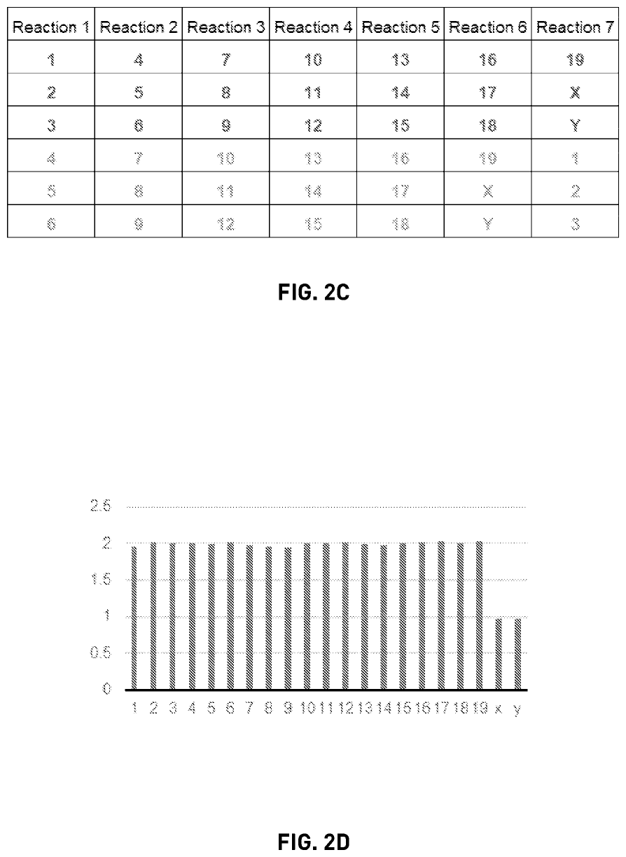

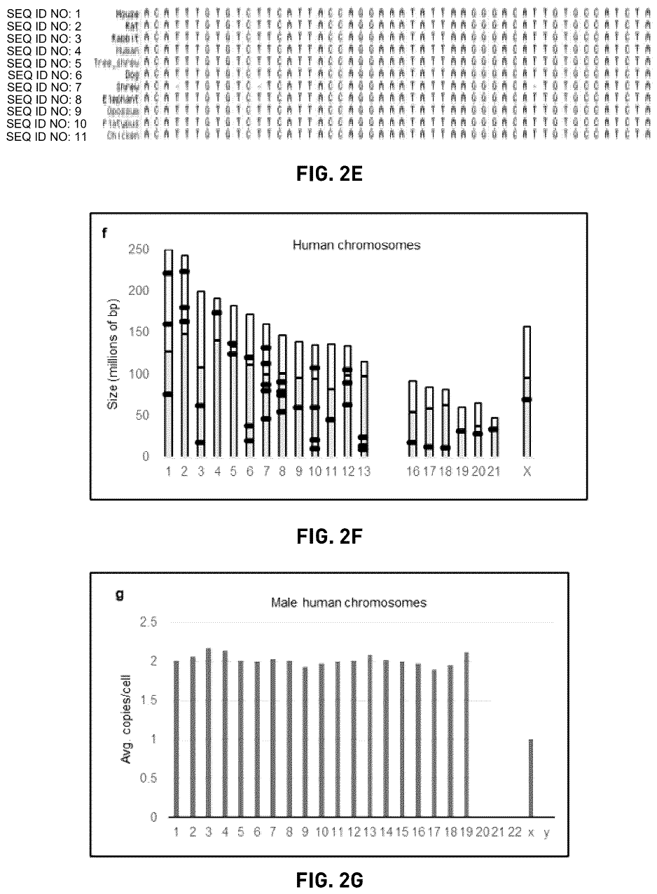

In accordance with the methods described here, each digital PCR reaction may contain multiple chromosomal primer pairs, and therefore will produce multiple amplification products, for example from 6-12 or more amplification products. Any suitable method for quantification of multiple different amplification products may be used, for example ratio-based multiplexing or amplitude multiplexing with additional fluorescent channels. The technique referred to as “ratio-based multiplexing with incomplete cluster designation”, or “ratio-based multiplexing” is exemplified by the examples here and is used to quantify the amount of each chromosome amplified by each chromosomal primer pair. Ratio-based multiplexing is described in detail in Whale et al., Biomol Detect Quantif, 2016. 10: p. 15-23. Briefly, this technique requires the single-positive clusters to be spread out in a manner such that the single-positive clusters are distinct from other single-positive and multi-positive clusters. However, the multi-positive clusters may overlap with other multi-positive clusters. When this is achieved, all calculations are made from the negative and single-positive clusters, while the multi-positive clusters are ignored. The methods described here utilize different detectable labels for each primer pair of a chromosomal set, for example FAM and HEX labelled primers, to enable full control of positive cluster placement in two dimensions. Using this technique, primer/probe sets can be multiplexed at high levels, for example 6-12 primer pairs per well, where each primer pair is directed to a template sequence of a target chromosome and at least two primer pairs are directed to different template sequences of the same target chromosome. This multiplexing strategy allows for the detection of aneuploidy using substantially fewer different reactions, for example in as few as 7 different reactions, i.e., 7 different wells of a microtiter plate, for a murine-derived cell line as illustrated in FIG. 2C. In this example, each reaction contains six primer pairs and each of the 21 murine chromosomes is amplified twice. Since aneuploidy is not usually extensive, the methods described here assume that the majority of chromosomes (except sex chromosomes) will exist as 2 copies/cell. Therefore, the ratios of the amounts of the two different amplification products for each target chromosome are compared to determine if there is gain or loss of a chromosome. A ratio of 2 indicates no aneuploidy. This multiplexing strategy drastically decreases cost, sample input, and time compared to other methods for detecting aneuploidy and can therefore be utilized routinely to monitor the quality of cell lines.

Ratio-based multiplexing is not frequently used because it can lead to a loss of precision, particularly when one of the multiplexed targets has a relatively high concentration compared to the other targets in the same reaction. However, this is not a concern for aneuploidy screening because all target chromosomes will exist in relatively similar ratios in the cells, e.g., 0, 1, 2, 3, 4 copies/cell. In addition, after developing an in silico model to boot-strap confidence intervals, it was determined by the present inventors that loading about 600 to 6000 cells per well would minimize variance when multiplexing 6-8 different targets in the same well.

Another advantage of this technique is it that heavy multiplexing reduces the need to run internal controls to account for well-to-well concentration variability. This is because changes in aneuploidy for just 1 or 2 targets will not greatly affect the median concentration measured from all targets in a given well. Therefore, the median concentration of each reaction can be compared to other reactions, and adjustments can be made to account for well-to-well concentration variation if needed.

In addition to ratio-based multiplexing, other multiplexing methods could be used, for example amplitude multiplexing with additional fluorescent channels.

In embodiments, the target chromosomal region of each target chromosome selected for amplification is highly conserved among mammalian species such as mouse, rat, and human; or at least among primate species, including humans and non-human primates. In the example below, the present inventors designed the aneuploidy primers/probes to bind highly conserved regions of the mammalian genome. This has two advantages. First, it minimizes the frequency for which unexpected genetic variations (SNPs, mutations, etc.) will impact the assay. Second, it enables adaptability of this assay to other species. For example, an ultra-conserved region in mouse chromosome 1 will also bind rat chromosome 5 and human chromosome 8. In embodiments, the invention provides a library of primer/probes targeting ultra-conserved regions of the mammalian genome and related compositions and methods utilizing the to provide aneuploidy assays for various mammalian species, including, for example, mouse, rat, dog, humans, and non-human primates.

### Cell Line Verification

The present invention provides methods of verifying the identity of a given cell line utilizing ddPCR-based analysis of single-nucleotide polymorphisms (SNPs) based on methodology described by Whale et al., Fundamentals of multiplexing with digital PCR. Biomol Detect Quantif, 2016. 10: p. 15-23. Whale et al describe ddPCR single nucleotide discrimination (ddPCR-SND) assays for measuring the concentration of SNP variants and determining the corresponding haplotype. These assays can also be multiplexed using ratio-based multiplexing. According to the methods described here, by analyzing the haplotype of multiple SNPs, a genetic fingerprint can be developed for each cell line of interest. This permits not only identification of a contaminating cell line, but also allows for quantification of the degree of contamination.

In embodiments of this method, a database such as that maintained by the 1000 Genomes Project, is used to identify dozens of single nucleotide polymorphisms (SNPs) that are evenly distributed in all human populations. Such SNPs are ideal for DNA fingerprinting and thus cell-line authentication. By selecting SNPs in specific chromosomal locations, ddPCR's absolute method of quantification can be used to simultaneously determine an SNP fingerprint for a particular cell line and its chromosome number, thus enabling the aneuploidy screen and cell line verification to be performed in a single assay. Accordingly, in embodiments, a set of SNPs is selected for detection such that each chromosome of the genome of the target cell line comprises at least two SNPs, preferably located at opposite distal ends of the chromosome from each other. In accordance with this embodiment, the SNP-based cell-line authentication assay can simultaneously be used to determine aneuploidy.

An exemplary assay layout is shown Table 1. In the first six reactions, 18 SNPs (on different chromosomes) can be identified to give a unique fingerprint that exists in 1/50 million people (0.375¹⁸). This is used to authenticate a cell line and will also detect contaminating cells. These same six reactions will simultaneously report the copy number variation among their 18 corresponding chromosomes. The next 4 reactions can be multiplexed to detect copy number variation among an additional 30 chromosomal regions. Thus, a total of 48 chromosomal regions can be counted, for which each chromosome is counted twice. Note this aneuploidy/authentication analysis is achieved using only 10 reactions. Another two reactions could be used to look for common mutations and Mycoplasma/bacterial infection, using the assay methods discussed below. Many other assay layouts could be employed to optimize for fewer reaction or more comprehensive screening.

TABLE 1
Exemplary assay layout

| Reaction # | Purpose | | Multiplexing | Target | Type |
|---|---|---|---|---|---|
| 1 | Authentication | Aneuploidy | 3 | SNP1-3; Chromo 1, 2, 3 | SND/CNV |
| 2 | Authentication | Aneuploidy | 3 | SNP4-6; Chromo 4, 5, 6 | SND/CNV |
| 3 | Authentication | Aneuploidy | 3 | SNP7-9; Chromo 7, 8, 9 | SND/CNV |
| 4 | Authentication | Aneuploidy | 3 | SNP10-12; Chromo 10, 11, 12 | SND/CNV |
| 5 | Authentication | Aneuploidy | 3 | SNP13-15; Chromo 13, 14, 15 | SND/CNV |
| 6 | Authentication | Aneuploidy | 3 | SNP16-18; Chromo 16, 17, 18 | SND/CNV |

| | | | | |
|---|---|---|---|---|
| 7 | Aneuploidy | 8 | Chromo 19, 20, 21, 22, X, Y, 1, 2 | CNV |
| 8 | Aneuploidy | 8 | Chromo 3, 4, 5, 6, 7, 8, 9, 10 | CNV |
| 9 | Aneuploidy | 7 | Chromo 11, 12, 13, 14, 15, 16, 17 | CNV |
| 10 | Aneuploidy | 7 | Chromo 18, 19, 20, 21, 22, X, Y | CNV |
| 13 | Common mutations | 6 | P53 mutations | SND |
| 12 | Mycoplasma infection | 3 | Mycoplasma/Bacteria/Mammal | SND |

### Detection of Common Mutations

Numerous mutations can arise during in vitro cell culture, however some hotspots are recurrent such as R175H in human TP53. These can be detected using custom designed or commercially available primer sets, for example the EGFR 6-color Crystal Digital PCR KIT from Stilla Technologies.

### Mycoplasma Detection

Ultra-conserved regions of bacterial DNA can be identified for which Mycoplasma DNA differs by a single nucleotide. ddPCR-SND assays can be designed to target this region, and measure total Mycoplasma DNA and total bacterial DNA. This ddPCR-SND assay can be multiplexed with an ultra-conserved region in mammalian (or animal) DNA, enabling simultaneous detection of Mycoplasma, bacteria, and mammalian cells. This assay can be used to quantify the extent of Mycoplasma and other bacterial infections by analyzing the DNA extracted from cultured cells or media.

Without such quantification, PCR-based Mycoplasma assays are prone to false positives, particularly due to single molecule carry over from a previous positive test. Additionally, current assays do not quantify the level of infection per cell, which could be useful for assessing efficacy of treatment and clinical testing. Finally, current genetic-based Mycoplasma tests are optimized for a very specific genetic sequence and could give false negatives from trace chemical PCR inhibitors (e.g., ethanol, heparin) or small future mutations. The present methods overcome these problems in three steps. First, by using digital PCR, the present assay is more resistant to chemical inhibitors of the PCR reaction. Second, the selection of an ultra-conserved element in mammalian DNA serves as a reference to quantify the amount of Mycoplasma genomes per mammalian cell. Finally, by using a single-nucleotide-discrimination assay, the methods described here are more tolerant to future mutations, as discussed below.

Mycoplasma and other bacteria have regions of DNA with some similarity. Trace amounts of bacterial DNA is pervasive in many lab reagents, thus Mycoplasma assays must not be cross-reactive if they wish to have high sensitivity. Therefore, traditional PCR-based Mycoplasma assays employ carefully designed primers to only amplify the intended target sequence and not amplify non-target sequences. However, when only a few mismatches separate target from non-target sequences, PCR conditions (i.e., annealing temp, extension time, cycles, etc.) may only work in a narrow range. In addition, primers with a few mismatches eventually mis-prime the non-target sequence when run for extended cycles. Once this occurs, the primer sequence is incorporated into the amplicon and subsequent PCR cycles amplify at high efficiency, leading to false positives. In contrast, mismatches in probes are not incorporated into amplicons, and therefore do not lead to false positives from mis-priming. The methods described here exploit these properties of PCR by utilizing degenerate or promiscuous primers to amplify a genetic segment that exists in nearly all bacterial genomes. These primers tolerate a few mismatches because the reactions are run for a high number of cycles. The probes, however, are used to distinguish Mycoplasma from other bacteria, for which occasional mis-annealing will not affect the outcome of the assay. Collectively, this system enables a more robust assay that uniquely detects and differentiates Mycoplasma and other bacterial infections. In addition, future point mutations that may occur in primer-binding regions will have minimal effect, while future mutations in the probe-binding regions are less likely due to the high conservation. However, even if a small mutation does occur in the Mycoplasma probe-binding region, the cluster position may slightly shift but it will still be detected at least within the bacterial cluster.

In embodiments, the degenerate or promiscuous primers amplify a genetic segment that exists in two or more bacterial genomes of a genus selected from the group consisting of Acholeplasma, Bacillus, Escherechia, Mycoplasma, Pseudomonas, Salmonella, Staphylococcus, and Streptococcus. In embodiments, the bacteria are selected from two or more of Acholeplasma laidlawii, Bacillus thuringiensis, Bacillus anthracis, Escherechia coli, Salmonella enterica, Staphylococcus auereus, Streptococcus plurextorum, Pseudomonas aeruginosa, Mycoplasma hominis, Mycoplasma bovis, Mycoplasma arginine, Mycoplasma hyorhinis, Mycoplasma fermentans, and Mycoplasma orale.

### Kits

The present invention also provides kits including one or more primer sets, selected from an Aneuploidy Set, an Authentication Set, a Mutations Set, and a Bacteria Set. In embodiments, the kit may include two or more of the aforementioned primer sets.

In embodiments, the Aneuploidy Set comprises a plurality of chromosomal primer pairs where each chromosomal primer pair is directed to a different template DNA sequence on a target chromosome in the genome of a target species, such as a human, mouse, rat, pig, goat, dog, cow, or other mammalian species, or a non-mammalian species such as yeast or an insect. The template sequences for each target chromosome may be located at opposite distal ends of the target chromosome from each other. The chromosomal primers may be unlabeled, or the primers may contain a detectable label. The kit may also comprise a plurality of DNA probes, each probe complementary to a region of a target chromosome between a chromosomal primer pair, each probe containing a detectable label. In embodiments, the number of chromosomal primer pairs, and optional DNA probes, is equal to at least twice the number of target chromosomes, such that each chromosomal primer pair or each primer-probe set is directed to a different template sequence on a target chromosome, the template sequences being located in different chromosomal regions, preferably toward opposite distal ends of the target chromosome. In accordance with this embodiment, the chromosomal primer pairs and/or primer-probe sets will provide two amplification products for each chromosome in a diploid genome such that each chromosome will be counted twice.

The Authentication Set comprises primer pairs and/or primer-probe sets targeted to a plurality of SNPs, wherein the plurality of SNPs is selected to uniquely identify a target cell line and optionally to identify one or more contaminating cell lines. In embodiments, some SNPs of the plurality may be selected to be located in different chromosomal regions, such that at least some of the primer-probe sets of the Authentication Set may be used as chromosomal primer pairs in the Aneuploidy Set. In embodiments, some SNPs of the plurality may be selected based on their distribution in a population, such as an animal strain, including e.g., a mouse or rat laboratory strain, or a human population, or across multiple human populations, for example SNPs having a relatively even distribution in certain ancestral, geographical, and/or ethnic populations, thereby enabling their use as unique “fingerprints” to identify cell lines derived from the same population. In embodiments, some SNPs of the plurality may be selected for their unique appearance in specific cell lines that commonly contaminate other cells lines, such as HEK and HeLa cell lines.

The Mutations Set comprises primer pairs and/or primer-probe sets targeted to detect single nucleotide mutations in commonly mutated genes, such as p53, or in a genomic region that is known to incur mutations during passage of cell lines. Exemplary p53 mutations include G245C, G245S, R248W, R248Q, and R175H.

The Bacteria Set comprises a set of degenerate primers targeted to a genomic region conserved across bacterial genomes as well as two primer-probe sets, the first adapted to distinguish a single nucleotide difference between Mycoplasma and most non-Mycoplasma bacteria and the second adapted to detect a conserved sequence in all mammalian DNA, such that the level of Mycoplasma/bacteria infection can be determined by quantifying the amount of amplification product detected by each primer-probe set.

In embodiments, the kit comprises a Mixed Set of primer pairs and/or primer-probe sets effective to perform two or more of the assays described herein simultaneously. In embodiments, the Mixed Set comprises a single set of primer pairs and/or primer-probe sets effective to perform a cell line authentication assay and at least a portion of an aneuploidy screen. In accordance with this embodiment, the Mixed Set may comprise primer pairs and/or primer-probe sets suitable for amplifying a plurality of SNPs located on a plurality of different chromosomes, wherein the plurality of SNPs is selected for their uniform distribution across a population, for example a human population or a given mouse or rat strain, such that the plurality of SNPs provides a unique fingerprint for a given cell line derived from an individual of the population, e.g., where the cell line is human, or an individual of the given mouse or rat strain. In accordance with this embodiment, the Mixed Set may be used in a method to simultaneously authenticate a cell line and detect contaminating cells.

In each of the following examples, each ddPCR reaction was prepared and analyzed with the QX200 ddPCR system (BioRad, Hercules, CA) per BioRad's standard recommendations for use with their ddPCR™ Supermix for Probes (No dUTP) unless otherwise stated. All reactions were mixed to 25 ul and contained up to 10 ul of DNA prepared from column purified extracts or crude lysate. For droplet generation, 20 ul were loaded into the droplet generator cassette in groups of eight per BioRad's protocol. Thermocycler conditions: 95° C.×10 minutes; 50 cycles of 94° C.×30 sec and 60° C.×60 sec; 98° C.×10 minutes; hold at 4° C. The average number of partitions after droplet generation was ˜15,000. Data analysis was performed with QuantaSoft Analysis Pro version 1.0.596 (BioRad, Hercules, CA).

### Multispecies Chromosome Counting (Aneuploidy Screen)

For proof-of-concept, primer/probe sets were designed to target each mouse chromosome twice. Thus, 42 sets were designed corresponding to 19 autosomes and 2 sex chromosomes of the murine genome. FIG. 2A illustrates preferred placement two sets per chromosome, each located in distal ultra-conserved regions of the same chromosome. This placement was possible for all chromosomes except the Y chromosome, for which two primer/probe sets were designed to target different regions on one end of the chromosome. Six primer/probe sets were included per well in a single multiplex reaction and fluorescence was detected simultaneously in two channels to detect the two different fluorescent labels used in each primer/probe pair, as shown in FIG. 2B. Thus, in FIG. 2B, the amplification products for Chromosome (“Chr”) 4 and Chr 5, (202, 204) are detected in the FAM channel while Chr 8 (210) and Chr 7 (212) are detected in the HEX channel. While the signals for Chr6 (206) and Chr9 (208) overlap in the FAM and HEX channels, they are sufficiently different to be distinguishing in the 2D plot. This enabled the counting of all chromosomes twice in only seven reactions as represented graphically in FIG. 2C. For example, in Reaction 1, three of the six primer/probe sets carrying a first fluorescent label amplify one of the ultra-conserved regions in each of chromosomes 4, 5, and 6. In Reaction 2, three of the six primer/probe sets carrying a second fluorescent label amplify a second ultra-conserved region in each of the same chromosomes 4, 5, and 6. Where the cells are substantially diploid, the ratio of the amplification products for each of chromosomes 4, 5, and 6 in Reaction 1 and Reaction 2 will be about 2. Any chromosome having a ratio more or less than 2 would be called as aneuploid. FIG. 2D shows the results of an assay performed with normal diploid male mouse DNA, correctly reporting the number of each chromosome as 2 for chromosomes 1-19 and 1 for each of the X and Y chromosomes.

Where the majority of primer/probe sets are designed to target ultra-conserved regions they can serve a dual purpose, that is for amplification of homologous regions in other species. FIG. 2E graphically illustrates representative alignments of such a highly conserved region in (from top) mouse, rat, rabbit, human, tree shrew, dog, shrew, elephant, opossum, platypus, and chicken. To illustrate the versatility of this approach, the 42 primer/probe sets designed for male mouse were used to amplify male human DNA. Most primer/probe sets found homologous regions in the human genome, which were mapped to the corresponding human chromosomes, as shown graphically FIG. 2F. The assay successfully counted 20 out of 24 human chromosomes at least once and counted 19/24 human chromosomes twice, as shown in FIG. 2G. This demonstrates how a library of primer/probe sets designed to target ultra-conserved chromosomal regions across different species could be used to detect aneuploidy among several species. This would result in considerable efficiencies because there would not be the need to design and synthesize new primer/probe sets for each chromosome of each species.

### Cell-Line Authentication

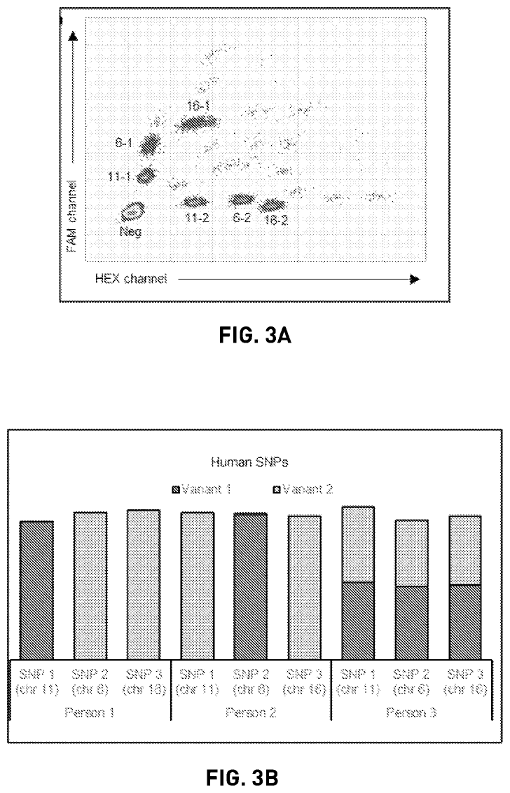

As proof-of-concept, three human SNPs were selected on chromosomes 11, 6, and 16. ddPCR-SND assays were designed for each SNP and multiplexed in a single well. Representative dual fluorescence imaging results are shown in FIG. 3A. Similar multiplexing was used as described in relation to multi-species chromosome counting above. When analyzing DNA extracted from three different people, each person had a unique fingerprint as depicted graphically in FIG. 3B. In addition, each person had two chromosomes per cell for the three targets. If desired, additional SNPs could be added and used to count additional chromosomes thereby providing a more unique fingerprint for each individual.

### Mycoplasma/Bacteria Detection

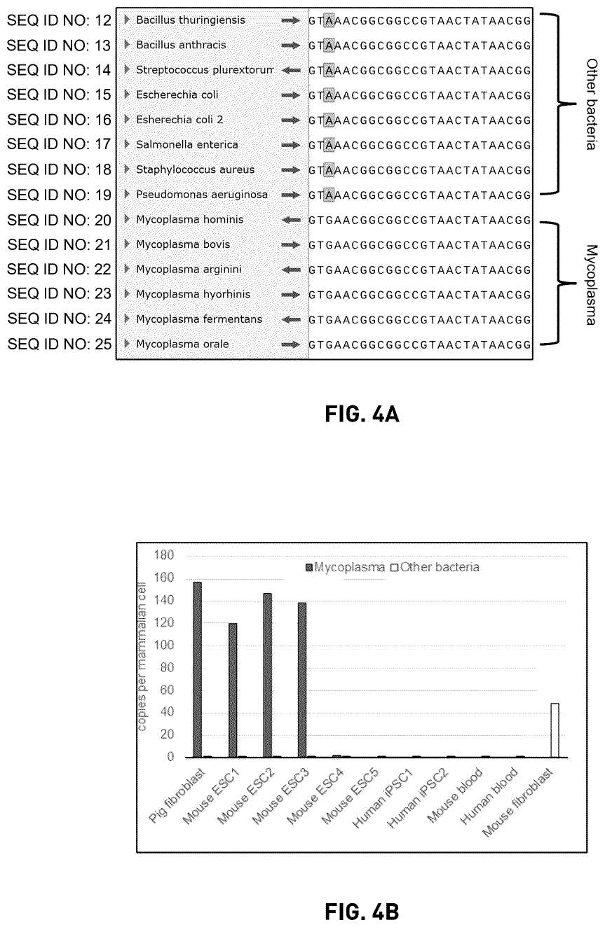

To validate this approach for Mycoplasma detection, or more generally for detection of bacterial contamination, a region of Mycoplasma ribosomal DNA was found that is conserved in most Mycoplasma and differs by a single nucleotide in most other bacteria. Alignments are shown schematically in FIG. 4A. A ddPCR-SND assay was designed around this region. Another primer/probe set was designed to detect mammalian DNA. The primer/probe sets were multiplexed and used to analyze DNA extracted from 10 cell lines from different species, four of which were known to be Mycoplasma positive. The assay worked as expected, with the Mycoplasma positive cells lines reporting an average of ˜140 Mycoplasma genomes per human cell. The results are shown graphically in FIG. 4B. A separate mouse fibroblast cell-line with an E. coli infection tested positive for bacteria, but not Mycoplasma (data not shown).

As proof-of-concept for detecting recurrent mutations in cultured cells, a ddPCR-SND assay was designed to quantify this R175H mutant. In a cell line with this mutation, dilution series were performed with wild-type DNA, and the proportion of mutant DNA was correctly detected (data not shown).

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

While the invention herein disclosed has been described by means of specific embodiments and applications thereof, numerous modifications and variations could be made thereto by those skilled in the art without departing from the scope set forth in the claims.

It will be appreciated that the present invention is set forth in various levels of detail in this application. In certain instances, details that are not necessary for one of ordinary skill in the art to understand the invention, or that render other details difficult to perceive may have been omitted. The terminology used herein is for the purpose of describing particular embodiments only and is not intended to be limiting beyond the scope of the appended claims. Unless defined otherwise, technical terms used herein are to be understood as commonly understood by one of ordinary skill in the art to which the disclosure belongs.

It should be understood that, as described herein, an “embodiment” (such as illustrated in the accompanying Figures) may refer to an illustrative representation of a process or article or component in which a disclosed concept or feature may be provided or embodied, or to the representation of a manner in which just the concept or feature may be provided or embodied. However such illustrated embodiments are to be understood as examples (unless otherwise stated), and other manners of embodying the described concepts or features, such as may be understood by one of ordinary skill in the art upon learning the concepts or features from the present disclosure, are within the scope of the disclosure. Thus, it is intended that the present subject matter covers such modifications and variations as come within the scope of the appended claims and their equivalents.

The presently disclosed embodiments are to be considered in all respects as illustrative and not restrictive, the scope of the claimed subject matter being indicated by the appended claims, and not limited to the foregoing description or particular embodiments or arrangements described or illustrated herein.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” means that the value may vary by +/−10%.

The term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The term “complement,” refers to a nucleotide (e.g., RNA or DNA) or a sequence of nucleotides capable of base pairing with a complementary nucleotide or sequence of nucleotides. Complementarity is determined by the ability of an associated nitrogenous base of a nucleotide, also referred to as a “nucleobase” or simply a “base”, to hydrogen bond with the nitrogenous base of a different nucleotide, e.g., a nucleotide on a different nucleic acid. This interaction may also be referred to as “base pairing”. The base adenine binds to thymine or uracil and the base guanine binds to cytosine. Adenine may therefore be referred to as the complement of thymine or uracil and guanine may be referred to as the complement of cytosine, and vice versa. Thus, a complement may include a sequence of nucleotides that base pair with corresponding complementary nucleotides of a second nucleic acid sequence.

The term “contacting” is used in accordance with its plain ordinary meaning and refers to the process of allowing at least two distinct species such as chemical compounds, biomolecules, and enzymes. to become sufficiently proximal to react, interact or physically touch.

The term “nucleic acid” refers to a polymer of nucleotides, e.g., deoxyribonucleotides or ribonucleotides, and may be used herein as shorthand for deoxyribonucleic acid (DNA) or ribonucleic acid (RNA).

The term “nucleoside” refers, in the usual and customary sense, to a glycosylamine including a nitrogenous base, also referred to as a “nucleobase”, and a five-carbon sugar, i.e., ribose or deoxyribose. Non limiting examples of nucleosides include cytidine, uridine, adenosine, guanosine, thymidine and inosine.

The term “nucleotide” refers, in the usual and customary sense, to the monomeric units of nucleic acids, each unit consisting of a nucleoside and a phosphate.

The term “base” as used herein with reference to sequences of nucleic acids refers to the nucleobase moiety of the nucleoside, e.g., cytosine, adenine, guanine, thymine, and uracil.

The terms “oligonucleotide,” “nucleic acid sequence,” and “polynucleotide” are used interchangeably and are intended to include a polymeric form of nucleotides covalently linked together that may have various lengths, either deoxyribonucleotides or ribonucleotides, or analogs, derivatives or modifications thereof. An oligonucleotide is typically composed of a sequence of nucleotides comprising nucleobases selected from adenine (A), cytosine (C), guanine (G), thymine (T), and uracil (U). Thus, the term “polynucleotide sequence” may refer to the alphabetical representation of a polynucleotide molecule; alternatively, the term may be applied to the polynucleotide molecule itself.

1. A method for detecting aneuploidy in a plurality of cells, the method comprising determining a ratio of an amount of each chromosome in a genome of the cells, the method comprising
contacting a sample comprising DNA from the cells with an enzyme mixture and one or more chromosomal primer pairs to produce a reaction mixture, wherein each chromosomal primer pair is directed to a different template DNA sequence on a target chromosome in the genome, preferably wherein the template sequences are located at opposite distal ends of the target chromosome from each other,
subjecting the reaction mixture to a digital PCR process including partitioning the reaction mixture and subjecting the partitioned mixture to PCR amplification to produce amplification products;
detecting the amplification products in the reaction mixture;
determining an amount of amplification product for each target chromosome; and
determining a ratio of the amount of amplification product for each target chromosome in the sample, wherein a ratio other than 2:2 for a target chromosome indicates aneuploidy.

2. (canceled)

3. The method of claim 1, wherein the amplification products of the chromosomal primer pairs are labelled with at least two different detectable labels.

4. The method of claim 1, wherein the one or more chromosomal primer pairs comprises from 1 to 12, from 3 to 6, from 6-9, or from 9-12 primer pairs, optionally wherein each primer pair in the reaction mixture is directed to a different target chromosome.

5. The method of claim 1, wherein the one or more chromosomal primer pairs contains a detectable label, optionally wherein two or more chromosomal primer pairs contain at least two different detectable labels.

6. The method of claim 1, wherein the one or more chromosomal primer pairs is unlabeled and the reaction mixture comprises one or more DNA probes, each comprising a detectable label, optionally wherein two or more DNA probes contain at least two different detectable labels.

7. (canceled)

8. The method of claim 1, wherein the enzyme mixture comprises a DNA polymerase, a source of divalent cations, a mixture of deoxynucleotide triphosphates (dNTPs), and a suitable buffering system.

9. The method of claim 1, wherein the method is performed by contacting a plurality of samples comprising DNA from the cells with a plurality of enzyme mixtures comprising one or more chromosomal primer pairs to produce a plurality of reaction mixtures.

10. The method of claim 9, wherein the contacting takes place in a plurality of separate containers, each container comprising a enzyme mixture and a plurality of chromosomal primer pairs.

11. The method of claim 10, wherein each chromosomal primer pair in the same container is directed to a template sequence of a different chromosome.

12. The method of claim 10, wherein the plurality of separate containers consists of from 5-15 containers.

13. The method of claim 10, wherein the plurality of separate containers comprise a microtiter plate.

14. The method of claim 1, wherein the template sequences of each target chromosome share at least 70%, preferably at least 90% sequence identity across human and non-human primate genomes; or at least 70%, preferably at least 90% sequence identity across human and mouse and/or rat genomes.

15. The method of claim 1, wherein two or more of the template sequences comprise at least one genetic mutation selected from a single nucleotide polymorphism (SNP) or a point mutation in a gene.

16. The method of claim 15, wherein the at least one genetic mutation comprises an SNP and the method comprises measuring a concentration of the SNP based on the amplification products in the reaction mixture and determining a corresponding haplotype for the cells.

17. The method of claim 15, wherein the at least one genetic mutation is a point mutation in a p53 gene, optionally wherein the genetic mutation is selected from the group consisting of G245C, G245S, R248W, R248Q, and R175H in a human p53 gene or corresponding mutation in a murine p53 gene.

18. The method of claim 1, wherein the method further comprises contacting a second sample comprising DNA from the cells with an enzyme mixture and a set of unlabeled primers directed to a sequence having high homology across two or more bacterial genomes to form a Mycoplasma detection reaction mixture.

19. The method of claim 18, wherein the set of unlabeled primers further comprises a first and second DNA probe, the first DNA probe comprising a first detectable label and the second DNA probe comprising a second detectable label.

20. The method of claim 18, wherein the first and second DNA probes comprise the same sequence except for a single nucleotide mismatch, wherein the single nucleotide mismatch distinguishes Mycoplasma from other bacteria.

21. The method of claim 18, wherein the method further comprises detecting Mycoplasma contamination in the cells based on detecting signals from the first and second DNA probes.

22. A kit comprising one or more primer pair and/or primer-probe sets selected from an Aneuploidy Set, an Authentication Set, a Mutations Set, and a Bacteria Set as described herein.
